(19) United States
(12) Patent Application Publication
(10) Pub. No.: US 2006/0253090 A1
(43) Pub. Date: Nov. 9, 2006

(54) Method and apparatus for facilitating evacuation and cleansing of a colonostomy bag

(76) Inventors: Arthur James Bradley, Red Bluff, CA (US); Donegal Bradley Bretzke, Red Bluff, CA (US)

Correspondence Address: CENTRAL COAST PATENT AGENCY, INC, 3 HANGAR WAY SUITE D, WATSONVILLE, CA, 95076, US

(21) Appl. No.: 11/256,301

(22) Filed: Oct. 20, 2005

Related U.S. Application Data
(60) Provisional application No. 60/678,897, filed on May 4, 2005.

(51) Int. Cl. A61F 5/44

(52) U.S. Cl. 604/334

(57) ABSTRACT

A system for top-down evacuating and rinsing of a user-mounted colostomy bag has a valve assembly affixed through one wall of the colostomy bag, the valve assembly having a first coupling interface and an interiorly protruding stem, at least one solution reservoir for containing an aqueous solution, and a second coupling interface attached directly to or indirectly to at least one of the at least one solution reservoir, the coupling interface for connecting the reservoir to the valve for communication of the aqueous solution from the reservoir to the bag.

### CROSS-REFERENCE TO RELATED APPLICATIONS

The present invention claims priority to a U.S. provisional patent application Ser. No. 60/678,897, entitled “Method and Apparatus for Evacuating and Cleansing a Colonostomy Bag” filed on May 4, 2005.

### BACKGROUND OF THE INVENTION

1. Field of the Invention

The present invention is in the field of medical waste management and pertains particularly to methods and apparatus for evacuating and cleaning user-worn colonostomy bags used to collect waste matter from patients.

2. Discussion of the State of the Art

In the field of post surgery management of biomedical fluids including waste matter, there is available in the art, a colonostomy, also referred to herein as ostomy or colostomy bag, that may be worn by a user and that is adapted to collect waste matter that has been surgically redirected from the normal path due to a re-routing of the colon during an operational procedure. Most commonly, the colostomy bag is used to collect solid waste from a patient who has undergone a surgical procedure on the colon and is used during in-patient treatment and, most importantly, after the patient has left the place of surgery until sufficient recovery time has elapsed so that a patient's normal function can be re-established. waste collected in the bag is manually coaxed toward a bottom opening in the bag, typically while the bag is attached to its host. Evacuation consists of simply forcing the excrement out of the bottom opening by hand. The bottom opening of the bag is foldable upon it self and closed with a clamp for reuse. Another challenge is cleaning the bottom opening so that it may be refolded and clamped. The entire process is demeaning to some users because of likelihood that their skin may come in contact with their own excrement. Likewise, current procedures do not enable the inside of the bag to be thoroughly cleaned while still attached to the host.

The inventor is aware of a waste evacuation pouch referenced herein as Flair Xtra™ that comprises an inner liner contained in the pouch. The pouch attaches to a user in a similar fashion as would a colostomy bag, however waste evacuates into the inner liner and not the pouch. A user must then remove the device and remove the inner liner manually in order to flush it and the contained waste matter down a toilet bowl. A problem with this technique is that it adds steps for a user and the user still must contend with spill-out and gaseous orders in some cases.

The inventor is aware of an colostomy pump device referenced herein as U.S. Pat. No. 6,840,923, issued on Jan. 11, 2005 to Milos Lapcevic, referred to hereinafter in this specification as Lapcevic. Lapcevic described an elongated cylindrical pump that may be attached directly to a stoma or re-routed bowel opening. Water is forced into the opening and waste matter is evacuated into the cylinder for subsequent waste treatment. The pump of Lapcevic is not applicable to evacuation or cleansing of an ostomy bag rather it is intended as a system for evacuating waste through a mammalian orifice. The pump of Lapcevic does not solve the evacuation problem created by users wearing evacuation bags attached to the stoma. Moreover, in all of the suggested embodiments of the pump of Lapcevic, the component requirements are quite extensive and the use of the pump in each embodiment is quite complicated and not suitable for the average user.

A second pump-type device is known to the inventor for cleaning receptacles of waste matter including colostomy bags. The device is reference herein as Australian patent AU-B-52792/93 published on Aug. 8, 1995 and entitled Colostomy Bag Plastic Bag and Beaker Cleaner. This device, like the one described further above depends on a double wall cylindrical construction maintaining an inlet for fluid injection and an outlet for waste evacuation. A problem with this technique is that a colostomy bag must be removed from the stoma before it may be cleansed. The apparatus also depends on an oblong mounting board for mounting over a toilet bowel where the evacuated waste will eventually be disposed. The device is complicated and requires much preparation and work in order to successfully evacuate a colostomy bag.

Yet another complex device for irrigating an ostomy opening is known to the inventor and is referenced herein as U.S. Pat. No. 6,595,971 issued Jun. 22, 2003 to von Dyck et al and entitled Ostomy Irrigation System. Von Dyck describes another complex system for ostomy irrigation by direct connection to the user's ostomy or stoma. The system is not applicable for colostomy bag evacuation or cleansing.

Still other systems are known to the inventor for irrigating the ostomy itself wherein a container or vessel, not a simple colostomy bag, is strapped to a user by belt and a tube is inserted through the vessel and into a patient (through the stoma) to irrigate the colon. One of these systems referenced herein as U.S. Pat. No. 2,540,777 entitled COLOSTOMY DEVICE, issued Feb. 6, 1951, includes an opening described as a nipple located top center of the vessel to which one may connect a water hose or tube to aid in flushing the interior of the vessel after irrigation of the colon has been performed using a primary irrigation tube. Another like device referenced herein as U.S. Pat. No. 2,438,273 includes an opening and associated tube of some length extending to the internal area of the vessel directed toward the stoma opening. Both of these devices have secondary flushing openings and tubes dedicated to helping flush the evacuated waste and water mix toward the bottom opening and into a toilet facility.

Neither of the devices mentioned above is a colostomy bag nor do they address the mobile use challenges of semi-permanent colostomy bags worn on a user while out in the field. It is important part of the recovery process that a user feel comfortable about the process of evacuating re-routed fecal matter whether that user happens to be in a hospital setting, at home, or out in public. In this mobile scenario, there is no colostomy irrigation process; rather the user ejects the matter into the bag naturally and then must contend with evacuation of the matter when the bag reaches a capacity for holding the material comfortably.

What is clearly needed in the art is a simple and modular system for aiding in evacuation of a colostomy waste collection bag and in maintaining some measure of cleanliness of the bag during continued use. A system such as this would facilitate mobile and stationary colostomy patients in such a manner as to alleviate much disdain and discomfort related to managing the evacuation ones own fecal matter.

### SUMMARY OF THE INVENTION

A system for top-down evacuating and rinsing of a user-mounted colostomy bag is provided. The system includes a valve assembly affixed through one wall of the colostomy bag, the valve assembly having a first coupling interface and an interiorly protruding stem, at least one solution reservoir for containing an aqueous solution, and a second coupling interface attached directly to or indirectly to at least one of the at least one solution reservoir, the coupling interface for connecting the reservoir to the valve for communication of the aqueous solution from the reservoir to the bag.

In a preferred embodiment of the method, the valve assembly is a unidirectional flow valve that is air tight when not coupled. In this embodiment, the exterior coupling interface is a valve body having an outside diameter thread formed thereon and the second coupling interface is a tube having an inner diameter thread formed thereon.

In one embodiment, the at least one solution reservoir is a syringe. In another embodiment, the at least one solution reservoir is a suspended intravenous bag. In yet another embodiment, the at least one solution reservoir is a toilet flush tank. In still a further embodiment, the at least one solution reservoir is a squirt bottle.

In one embodiment, the indirect attachment of the second coupling interface to the at least one solution reservoir is made by inclusion of a length of tubing between the connector and the reservoir. In this embodiment, the reservoir may be an intravenous bag or a toilet flush tank.

According to one aspect of the present invention wherein the reservoir is an intravenous bag, the system further includes a squeeze bulb for urging the aqueous solution forward and, a tubing clamp for stopping and starting gravitational occupation of aqueous solution through the length of tubing.

According to another aspect of the present invention a colostomy bag is provided. The colostomy bag includes at least two bag walls seamed together; and

a valve assembly permanently affixed through one of the bag walls, the assembly including an externally protruding coupling interface and an interiorly protruding valve stem.

In a preferred embodiment, the valve assembly couples to a system for delivering an aqueous solution into the bag while worn by a user. Also in a preferred embodiment, the valve assembly is a unidirectional flow valve that is airtight when not coupled. Also in a preferred embodiment, the externally protruding coupling interface is a valve body having an outside diameter thread formed thereon connectable to a second coupling interface having an inner diameter thread formed thereon.

According to a further aspect of the present invention, a method is provided for top-down evacuating and rinsing of a colostomy bag mounted to a user, the colostomy bag including a valve assembly permanently affixed through one of the bag walls, the assembly including an externally protruding coupling interface and an interiorly protruding valve stem including acts for (a) coupling a delivery system adapted to deliver an aqueous solution to the coupling interface; (b) delivering an amount of the aqueous solution into the bag through the valve assembly; (c) massaging the bag externally to break down solid matter into the solution; (d) unclamping an evacuation opening at the bottom of the bag; and (e) rinsing the bag by delivering an additional amount of aqueous solution into the bag through the valve.

In one aspect, in act (a), the delivery system is a syringe coupled to the valve assembly by threading. In a preferred aspect in act (b) the valve assembly is a unidirectional flow valve that is airtight when not coupled.

In one aspect, in act (a), the delivery system is a suspended intravenous bag indirectly coupled to the valve assembly via a length of tubing. In a variation of this aspect, the tubing includes a hollow-bodied squeeze bulb. In another aspect, in act (a), the delivery system is pump activated.

### BRIEF DESCRIPTION OF THE DRAWING FIGURES

FIG. 1 is a plan view of a colostomy bag according to prior art.

FIG. 2 is a plan view of a colostomy bag according to an embodiment of the present invention.

FIG. 3 is a partial view of colostomy bag of FIG. 2 according to an embodiment of the present invention.

FIG. 4 is a sectioned view of valve assembly 2007 of FIG. 3 taken along section line AA.

FIG. 5 is a block diagram illustrating a valve assembly process 5000 according to an embodiment of the present invention.

FIG. 6 is a partial view of colostomy bag 2000 of FIG. 2 illustrating syringe 3001 according to an embodiment of the present invention.

FIG. 7 is a plan view of a colostomy bag 7000 with an extended opening according to another embodiment of the present invention.

FIG. 8 is an elevation view of a user 8000 practicing colostomy evacuation and rinsing according to other embodiments of the present invention.

FIG. 9 is a process flow chart 9000 illustrating acts for evacuating and rinsing a colonostomy bag through a colonostomy bag valve according to an embodiment of the present invention.

### DETAILED DESCRIPTION

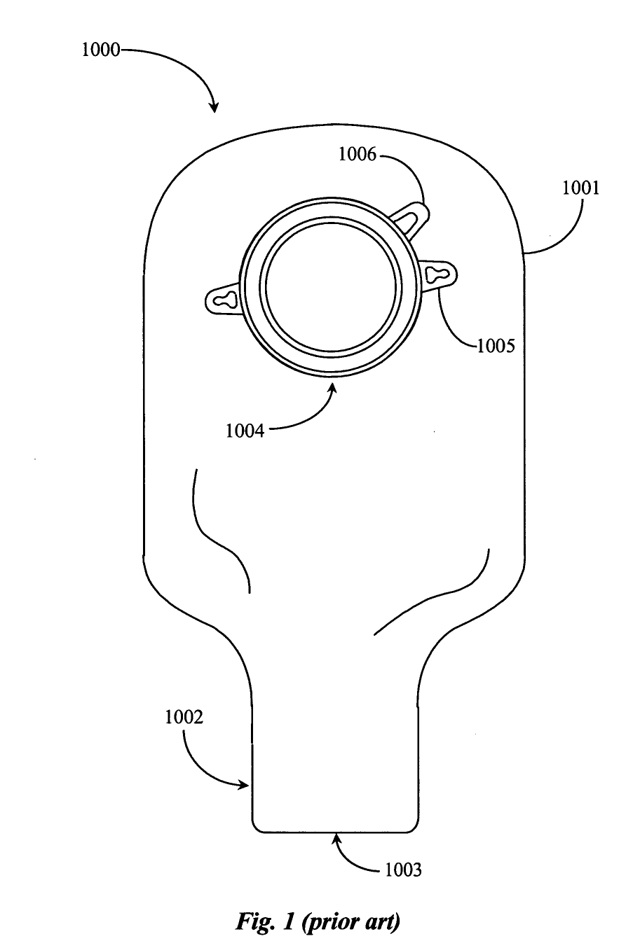

FIG. 1 is a plan view of a colostomy bag 1000 according to prior art. Colostomy bag 1000 is adapted as a temporary waste collection bag that is worn externally by a user and at some point discarded after some repeated use. More particularly, bag 1000 is adapted as an externally worn system for collecting solid waste from a surgery patient whom has had surgery on the colon and has had function of the colon temporarily, or in some cases, permanently re-routed. Bag 1000 is manufactured of a pliable and leak-proof polymer 1001. Material 1001 includes 2 opposing thin and pliable sheets of plastic and a soft material sheet (not illustrated) attached on one of the plastic sheets on a side facing the user when mounted. The opposing sides of bag 1000 may be glued together or fused via a heating treatment, or a combination of the two. The soft material sheet may also be glued in place and is provided so that the user may be more comfortable when wearing the bag. The soft material sheet serves no other function than to provide a comfortable interface against the skin of a user.

Colostomy bag 1000 is a discardable bag that is typically obtained in a package of several bags. Bag 1000 includes a stoma connector 1004 adapted to enable a user to connect bag 1000 to like apparatus (not illustrated) attached via tape or via other method over a re-routed stoma (surgical opening). Connector 1004 includes an alignment tab 1006 and snap flanges 1005. Connector 1004 is used to secure bag 1000 to the associated device attached to the patient's new opening. Connector 1004 has an opening centered therein, which is significantly large and adapted to allow solid waste to pass from the patient's colon and re-routed stoma and then into bag 1000.

Bag 1000 has a bottleneck 1002 formed therein at the bottom portion of the bag. Bottleneck portion 1002 includes a bottom opening 1003 that may be folded over upon itself in order to close the bottom opening of bag 1000 to prevent leakage. Typically, a clamping device (not illustrated) is provided for the purpose of closing the opening at the bottom of bag 1000.

As described above, colostomy bag 1000 is worn by a user and adapted to receive waste as it passes naturally from the user. After some time of use where waste has been collected by bag 1000, a user may desire to evacuate that bag of waste to free the bag for collecting subsequent waste from the user. As was described further above with respect to the background section and the discussion of state of the art, a user must manually evacuate the bag through the bottom opening and then re-clamp the bottom opening to again seal the bag for continued function. A user need not remove the bag when evacuating waste there from. After a few to several cycles, a user discards colostomy bag 1000 and installs a new one for continued use. As was described further above the process now in place for manual evacuation of this type of colostomy bag is both difficult to achieve without some difficulty and mess accorded to the user. Therefore a new type of bag and a new process is required to alleviate the existing problems.

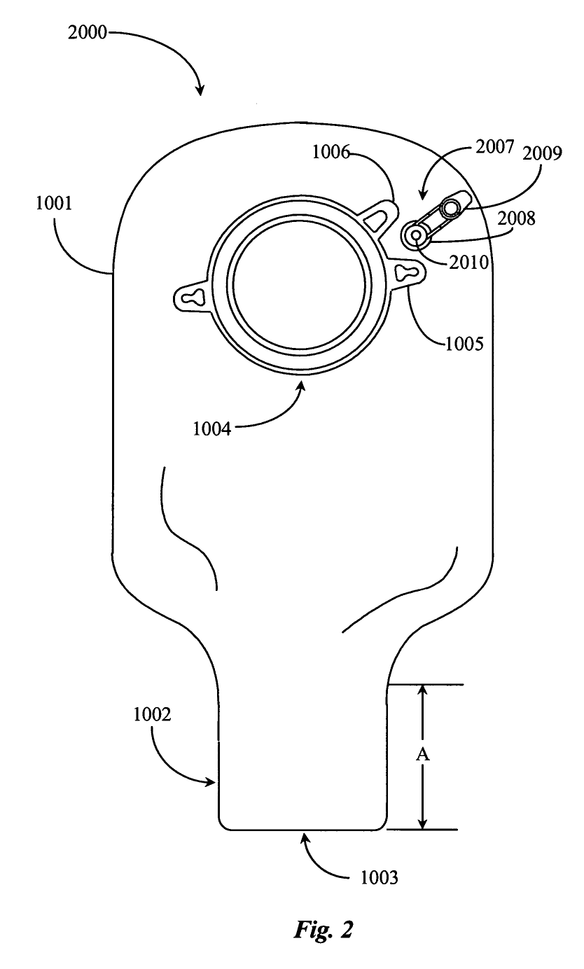

FIG. 2 is a plan view of a colostomy bag 2000 according to an embodiment of the present invention. Colostomy bag 2000 includes all of the components described with respect to prior-art colostomy bag 1000 described further above. Elements of colostomy bag 2000 that were previously described above and are not modified to practice the present invention shall retain the same element numbers and shall not be re-introduced.

Colostomy bag 2000 may be a modified version of colostomy bag 1000 without departing from the spirit and scope of the present invention. For example, bag 2000 may include connector 1004 including alignment tab 1006 and snap flanges 1005. Likewise, material 1001, bottle neck 1002, and bottom opening 1003. In this example, the length dimension A of bottle neck 1002 from radius to opening 1003 is approximately 2.5 inches, which is unmodified from the prior art version of the bag illustrated with respect to FIG. 1 above. However, it may be desired in some cases to provide a greater length for the described dimension for a purpose that will be detailed later in this specification.

A valve assembly 2007 is provided to and installed on colostomy bag 2000 at a strategic location on the same side of the bag supporting stoma connector 1004. Valve assembly 2007 is installed at a location to the right of and substantially above horizontal center of connector 1004 between alignment tab 1006 and one of snap flanges 1005 in this embodiment.

In one embodiment, valve assembly 2007 includes an outer valve body 2008, an inner valve passageway 2010, and an attached valve cap 2009. Valve assembly 2007 is installed, in a preferred embodiment, on the side of bag 2000 comprising one plastic sheet glued or otherwise attached to a soft material layer. This is the side that comes in contact with a user when worn. The opposite side of bag 2000 is a sheet of plastic. Material 1001 described above encompasses the 3 pliable sheets of material that are glued or otherwise joined along aligned peripheral edges to form the seamed colostomy bag.

Valve assembly 2007 may be installed by first creating a small opening through the soft material and underlying plastic sheet forming the user-facing side of the bag. Valve assembly 2007 may be inserted partially through the opening in the correct orientation, a stem portion thereof protruding into the inside of bag 2000. Valve assembly 2007 may be glued in place using a strong and fast drying epoxy or glue.

The purpose of valve assembly 2007 is to provide delivery access to the interior of bag 2000 while a patient is wearing it. The internal portion of the bag would otherwise be inaccessible accept for through opening 1003 if unclamped. Valve assembly 2007 may be provided of a flexible plastic or a hard plastic, or a combination of flexible and hard plastic. Valve assembly 2007 is adapted for coupling to a system or device capable of delivering an aqueous solution into the bag. In a preferred embodiment, the aqueous solution is used to help dissolve any solid waste matter collected in the bag to make it easier to evacuate. The solution can be one of or any mixture of water and one or more agents designed to clean, promote solubility, to disinfect, and so on.

In one embodiment, valve assembly 2007 is a modified “Lockport” valve, modified to enable the valve assembly to be used according to an embodiment of the present invention. Such a valve will be further detailed later in this specification.

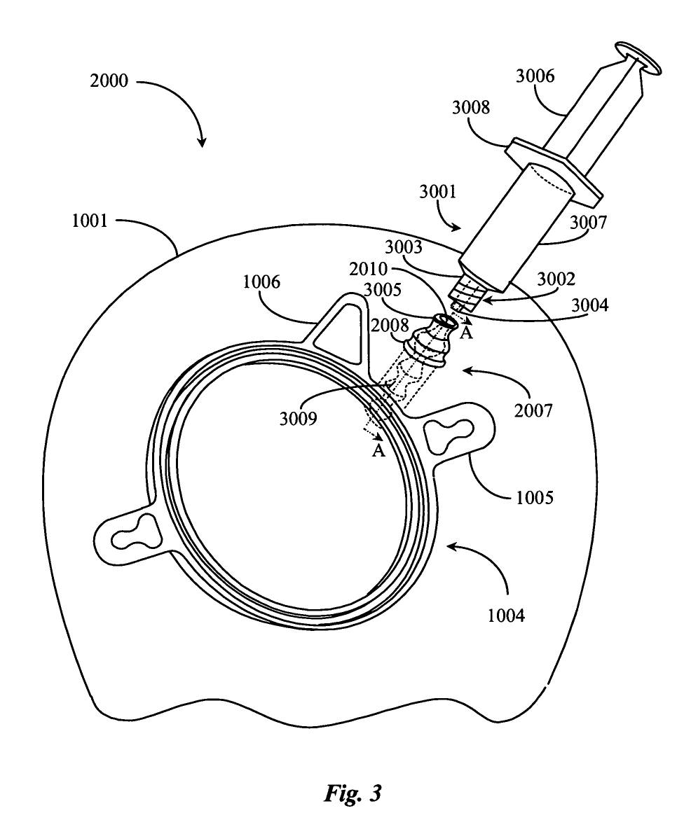

FIG. 3 is a partial view of colostomy bag 2000 of FIG. 2 according to an embodiment of the present invention. Valve assembly 2007 may be a modified version of a lockport valve in one embodiment without departing from the spirit and scope of the present invention. Valve assembly 2007 is available to the inventor as a Lockport valve used in the medical industry as a one-way valve for administering fluids or oxygen delivered through an intravenous (IV) system. The valve assembly must be modified for successful use in the present invention. The valve is also known as a needle-free valve port model number 2000EE, lot number 05025644, available through Alaris Medical Systems Inc. In this example, valve assembly 2007 represents a needle-free valve port or the Lockport referenced above. However, using a valve that is modified for use in the present invention is not specifically required in order to practice the present invention. In other embodiments, other types of valves may be used that do not require modification without departing from the spirit and scope of the present invention. Characteristics of the Lockport valve are desired specifically, a characteristic that prevents a reverse flow of matter or gas that may otherwise emanate from the interior of bag 2000 if a bi-directional valve were used.

Valve body 2008 includes an outside diameter (OD) threading 3005 about the periphery of valve body 2008 at the location of valve passageway 2010. An aqueous delivery system 3001 is provided and is adapted as a syringe for coupling to valve assembly 2007 in order to deliver an aqueous solution into colostomy bag 2000. Syringe 3001 has a body portion 3007 adapted to retain the solution for delivery. Body portion 3007 has a plunger base 3008 formed thereon at one end providing a plunger stop for a sliding plunger 3006. Body portion 3007 has a volume of at least 60 cubic centimeters (CCs), which is sufficient for injection of a generous amount of drawn fluid for evacuating bag 2000. Syringe 3001 and the components thereof may be manufactured of a polymer material typical for medical syringes. An annular rubber stopper (not illustrated) is typically provided at the enclosed end of sliding plunger 3006 provides normal syringe functionality including drawing of fluid into body 3007 and ejecting fluid from body 3007.

Syringe 3001 has a double tube nozzle 3002 including an outer nozzle tube 3003 and an inner delivery tube 3004. Inner delivery tube 3004 has an outer diameter of a dimension slightly smaller than the inner diameter (ID) of valve assembly opening 2010. Outer nozzle tube 3003 has a thread provided around the inner periphery of such a dimension so as to enable threading of nozzle 3002 onto and over valve body 2008. Passage 2010 is filled with special hollow body plug 3009 provided in the form of a resilient, elastic material such as a synthetic rubber or neoprene. Plug 3009 includes a short stem portion for blocking passage 2010 at the outer free end of valve body 2008. The short stem has a very small micro slit (not illustrated) placed vertically there through. The slit extends through the center of the solid material portion of plug 3009. The remainder of plug 3009 exhibits a hollow body characteristic. Valve assembly 2007, as described further above extends through one wall of bag 2000 to the inside of the bag as illustrated herein by a stem body 211 represented herein by hidden line. Stem body 2011 is adapted to enclose plug 3009 in such as way as to accommodate for the segmented shape of the hollow body portion of the plug. A small through opening is provided at the end opposite the materially solid end of plug 3009 that opens up into the hollow body portion of the plug.

Valve assembly 2007, via plug 3009, works to seal the valve when syringe 3001 is not coupled to it. Therefore in a sealed position plug 3009 is fully extended within stem body 211 and valve body 2008 thus sealing the valve at the opening 2010. The micro slit through the plug stem is so small that when collapsed (plug extended), it does not allow fluid or air to flow into or out of the valve stem. When syringe 3001 is threaded onto and over valve body 2008, delivery tube 3004 acts to displace the materially solid portion of plug 3009 inward into the stem body portion 2011 via compression. This action causes the micro slit to expand and open creating a passageway into the interior of plug 3009, which is hollow. Therefore, when valve assembly 2007 and syringe 3001 are fully coupled passageway 2010 is fully open and aqueous solution may then be passed from syringe 3001, through plug 3009 and into colostomy bag 2000. In other embodiments other coupling methods may be utilized instead of threading such as snap coupling or press fitting without departing from the spirit and scope of the present invention. In this example, threading syringe 3001 onto valve body 2008 provides a one way delivery system whereby aqueous solution may be delivered without any solid, liquid or gaseous contents within bag 2000 escaping through valve assembly 2007 to the outside. Other one-way compression valves are available and with some modification, could be adapted for the purpose of the invention.

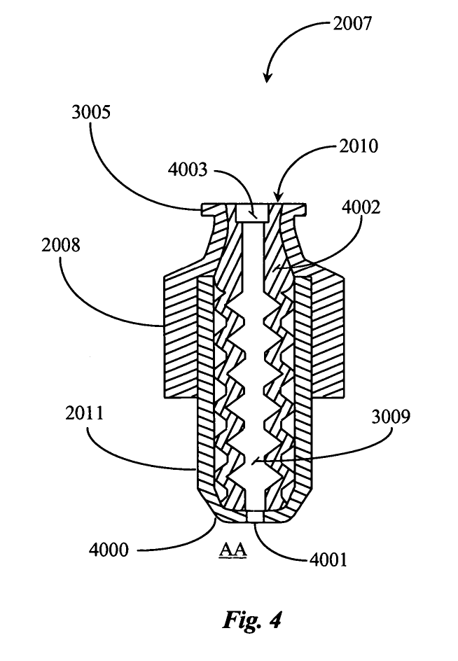

FIG. 4 is a sectioned view of valve assembly 2007 of FIG. 3 taken along section line AA. Valve 2007 includes valve body 2008 and valve stem 2011 as previously described above. Valve body 2008 includes threading 3005 and opening or passageway 2010. Valve stem 2011 includes an opening 4001, and a shoulder section 4000. Shoulder 4000 functions to constrain plug 3009 within valve stem 2011 at that end. Valve body 2008 is tapered toward opening 2010. The taper functions to constrain plug 3009 to within valve body 2008 at that end. In this way, plug 3009 is encapsulated within the valve assembly. In a preferred embodiment, valve assembly 2007 is glued into place on a colostomy bag. Application of a fast drying and flexible adhesive may be made at the general location of the junction of valve body 2008 and valve stem 2011 on the bag interior and on the bag exterior. Valve stem 2011 occupies the interior of the colostomy bag while the valve body is exposed to the exterior of the bag. Moreover, heat susceptive flanges may also be provided to facilitate a heat activated gluing such that the area immediately around the valve assembly is leak tight and strong enough for many use applications before discarding the bag.

In this example, valve assembly 2007 is modified from a Lockport valve by removing a female threaded connector by grinding the connector off just below shoulder portion 4000. However, in one embodiment a new or enhanced valve component may be molded without a female connector on the end opposite valve body 2008. In a preferred embodiment, valve assembly 2007 is a unidirectional valve. In this embodiment it is not required to cap the valve body as no materials or gas may escape the valve. The meat or material of plug 3009 is illustrated in section view as element 4002. It is noted that the accordion profile revealed in this section view provides the basis for expansion and compression within the cavity such that when plug 3009 is allowed to expand, it seals opening 2010 and prevents any back leakage through opening 2010. When plug 3009 is compressed, solution may be delivered through plug 3009 and through opening 4001.

In an alternate embodiment plug 3009 may be removed and openings 2010 and 4001 may be slightly enlarged to promote flow of aqueous solution from a gravity activated delivery system such as a funnel or a reservoir suspended at some height above a user evacuating and rinsing the inside of colostomy bag 2000.

Referring now back to FIG. 3, syringe 3001 is used to deliver an aqueous solution into bag 2000 through valve assembly 2007 while it is connected to a patient in order to first help to dissolve any solid waste matter collected before the bag is evacuated. The solution enters the bag with sufficient force and the flow of solution can be manually directed to engage different portions of the interior of bag 2000 as will be demonstrated later in this specification. A patient may physically massage the outside of bag 2000 after a delivery of solution to further aid in the breaking up of excrement. In some cases, more than one injection of aqueous solution may be desired.

After the solid waste has been dissolved or liquefied, the bottom opening of bag 2007 may be un-clamped and unfolded to allow for evacuation of the bag into a suitable repository. Subsequent flushing using additional aqueous solution can then be directed along the interior of the now-evacuated bag to rinse or cleanse the bag removing any residual waste matter. The process is performed while the patient is wearing the bag and evacuation may occur into a toiletry receptacle in an efficient manner.

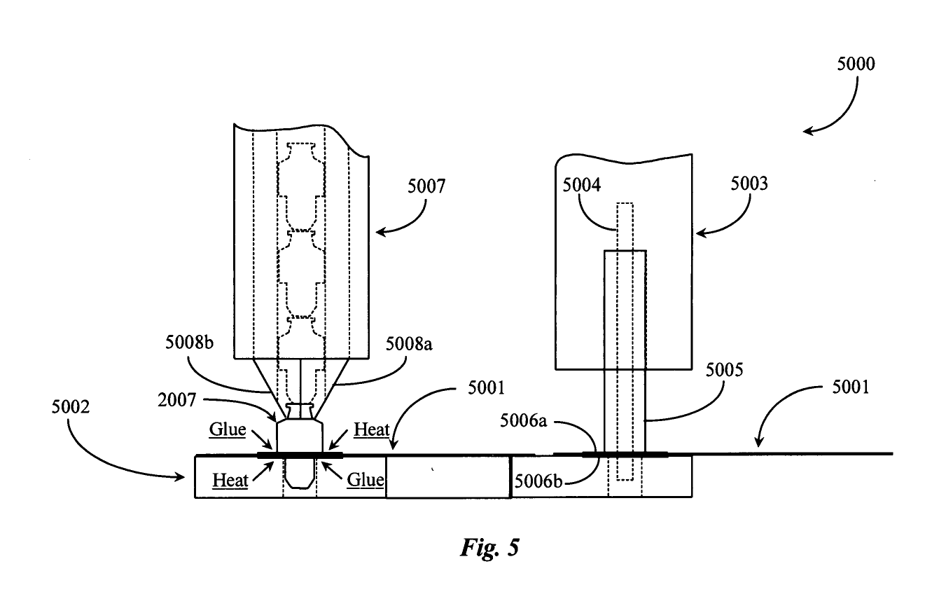

FIG. 5 is a block diagram illustrating a valve assembly process 5000 according to an embodiment of the present invention. Assembling valve assembly 2007 to a colostomy bag may be practiced before the bag is actually formed and sealed. A colostomy sheet 5001 comprising a ply of plastic and a ply of soft material may be seamed together to form one wall of the two-wall bag.

A work plate or support bench 5002 may be provided and adapted to receive sheet 5001 and to facilitate forward movement of the sheet to receive one or more than one automated treatment to facilitate assembly of the valve to the sheet at the targeted location. Work plate 5002 may be bored to accept a punch tool 5004 adapted to provide the opening for valve assembly insertion. Punch tool 5004 may be slidably mounted through the center portion of a stabilization bar 5005 adapted to stabilize or to hold the material in place around the punch-through location in sheet 5001. Bar 5005, including punch tool 5004 may both be mounted to a tooling housing 5003, which may in turn be removeably affixed or otherwise tooled to a process machine adapted to deliver enough downward force to drive punch tool 5004 through sheet 5001 in order to prepare the sheet for receiving a valve assembly.

The driving force of the machine may be hydraulic fluid, air pressure, electronic or even manual operation via a human or a robotic operator. In one embodiment, existing equipment used to fabricate the prior-art version of the colostomy bag can be easily modified to add the capability of inserting the valve assembly through and sealing the valve assembly onto the appropriate sheet comprising one side of the bag. In this example heat-activated glue flanges or washers 5006a and 5006b are placed in position and pressed onto sheet 5001 before an opening is provided through the sheet and perhaps the washers as well.

A tool housing 5007 is provided and adapted to present the appropriate tools to perform the process of placing the valve assembly through the punch-through opening made by punch 5005. In this example, housing 5007 supports an inner dispensing tube 5009 that is pre-loaded with valve assemblies. In one embodiment, dispensing tube 5009 may have opposing movable placement apertures 5008a and 5008b. Valve assembly 2007 may be robotically placed through the opening in the colostomy sheet and, in this case through the opposing heat activated glue washers. In one embodiment, dispensing tube 5009 may be retracted from housing 5007 and a second tool (not illustrated) may be inserted therein robotically to provide a press land area and heating surface with which to press and heat washers 5006a and 5006b on the upper surface. Heat may also be provided through work plate 5002 of a sufficient temperature to activate the washers to form a seal around the periphery of valve assembly 2007 on both sides of sheeting 5001.

One with skill in the art of automated and robotic systems will appreciate that there are many existing tooling sets and machines that can be used to install valve assemblies to material or to plastic sheeting in general. Some of these may be modified to manufacture the colostomy bag of the present invention in an economic fashion such that mass production may be achieved without great expense. For example, the tooling used at present to manufacture the colostomy bag without the valve may be modified to add the valve assembly to the bag.

In an alternate embodiment, valve assembly 2007 may be installed in a home setting by a user with the aid of a valve assembly kit consisting of the appropriate hand tools and glue for installing the valve. In this case, the colostomy bags may be purchased off the shelf and a user may modify them at will. In a preferred embodiment however, the bags are manufactured with the valve in place and ready for use.

In another embodiment, valve assembly 2007 may be placed onto a colostomy bag after the bag has been fully seamed, valve placement being a last step in the process. In still another embodiment, the valve assembly may be pressed and glued to one side of sheeting 5001 and then the sheet may be flipped over for gluing the other side. The advantage of special washers enables quick and measured sealing in an environment of automation. Work plate 5002 may be a stable work plate and housings 5003 and 5007 may be apertures that swing or rotate into place similar to tooling chucks used on some C&C machines. There are many possible tooling architectures that may be conceived without departing from the spirit and scope of the present invention such architectures ranging from completely manually operated tooling to fully robotic automated tooling. There are many possibilities.

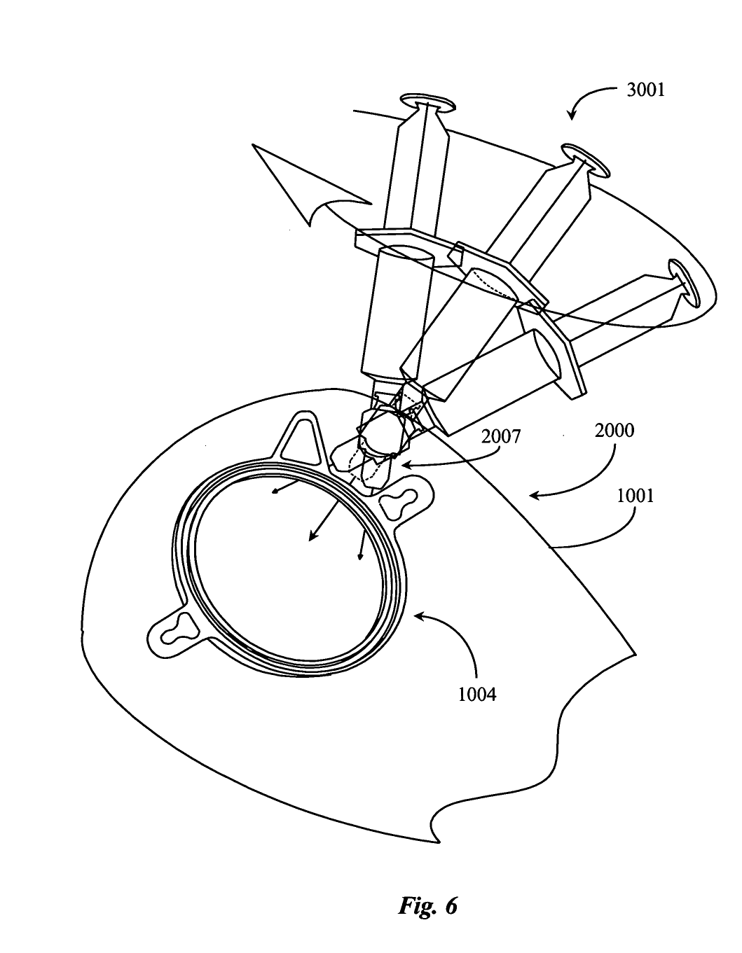

FIG. 6 is a partial view of colostomy bag 2000 of FIG. 2 illustrating syringe 3001 according to an embodiment of the present invention. Colostomy bag 2000 is coupled to syringe 3001 via valve assembly 2007 in this embodiment. It is noted herein that in active use, connector 1004 would be engaged to the appropriate connector around the stoma of a user. The visible side of bag 2000 in this view would present against the body of that user.

When the bag becomes full, the user may step into a bathroom, for example, take out syringe 3001 and draw water into it until it is fully drawn. The user may then thread syringe 3001 over assembly 2007 creating a unidirectional passageway for the solution. While injecting the solution in this manner, the user may change the directional positioning of syringe 3001 at will due to the flexible nature of colostomy bag 2000. A sample movement is illustrated herein by inclusion of hairline outlines depicting syringe 3001 in positions that are variant from the primary position. Syringe 3001 can be manipulated to trace a circle as is shown in this example by a large directional arrow. Likewise the user manipulates the syringe movement urging the syringe in the opposite direction from that direction illustrated here. Small directional arrows emanating from the valve assembly illustrate the current direction of the aqueous solution into bag 2000.

One with skill in the art will appreciate that syringe 3001 may be manipulated during use to direct a stream of solution to any inside area of colostomy bag 2000 due to the flexible nature of the valve adhesion to the bag surface. The exact directional range achievable is far greater than what is illustrated in this example. One particular advantage of the strategic location of valve assembly 2007 on bag 2000 is that the aqueous solution may be delivered in a manner that urges the contents toward the bottom opening of the bag for convenience and prevents splash-up that may occur attempting to rinse the bag from the vantage of the bottom opening only after it has been evacuated. Therefore, much work can be eliminated and no external equipment comes into contact with the bag contents. It is reiterated here that in a preferred embodiment, the method of the present invention is to first deliver aqueous solution to help “liquefy” solids that may be present aiding in gravity evacuation without requiring external massage of the bag to force solids down. Secondly, additional aqueous solution including disinfecting agents or the like may be used to treat any remaining residue after evacuation leaving a relatively clean bag interior for a next cycle.

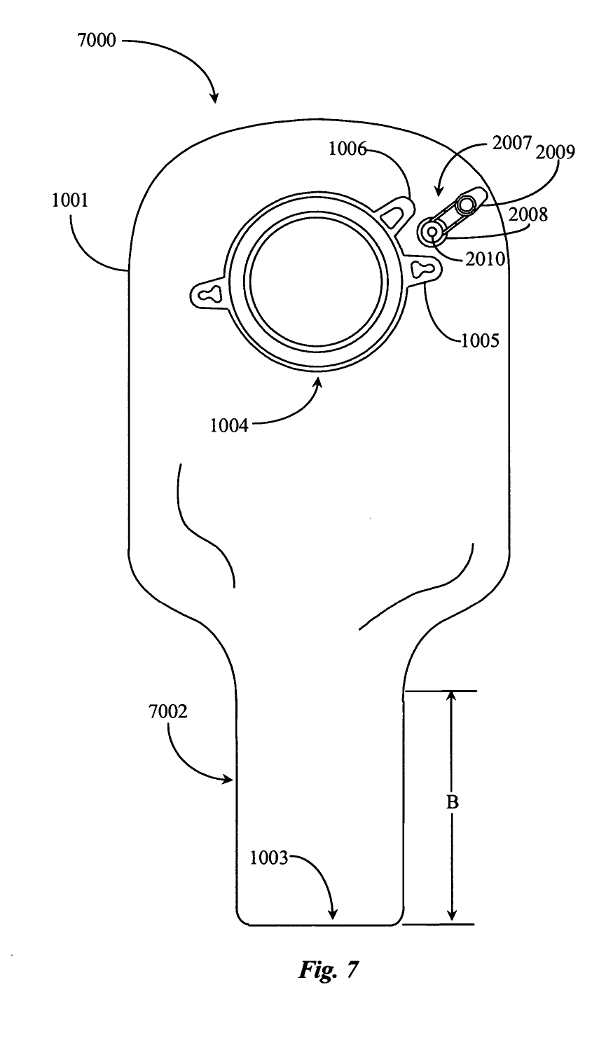

FIG. 7 is a plan view of a colostomy bag 7000 with an extended opening according to another embodiment of the present invention. Bag 7000 is analogous to bag. 2000 except for an enhancement that further facilitates mobile use and evacuation of the contents of the bag into a toilet facility or other waste receptacle. Elements illustrated in this example that are also present in the example of FIG. 2 shall retain their same element numbers and shall not be reintroduced herein unless they have been modified according to the present embodiment.

Colostomy bag 7000 has a much elongated bottleneck portion 7002 illustrated in this example as dimension B from radius to opening 1003. Dimension B may be greater than 2.5 inches up to about 4 inches in length. This enhancement facilitates top-down evacuation of colostomy bag 7000 directly into a toilet facility or other waste receptacle while a user is wearing the bag in a sitting position. In the case of dimension B, a user is better able to evacuate liquefied matter from the bag in a manner that ensures none of the matter is displaced or spilled out because of a short bottleneck. Bag 7000 is also rinsed from the top down so no fixtures need be inserted up through the bottom opening to clean or rinse the bag. Therefore, a longer opening facilitates user comfort without compromising the rinse ability of the bag.

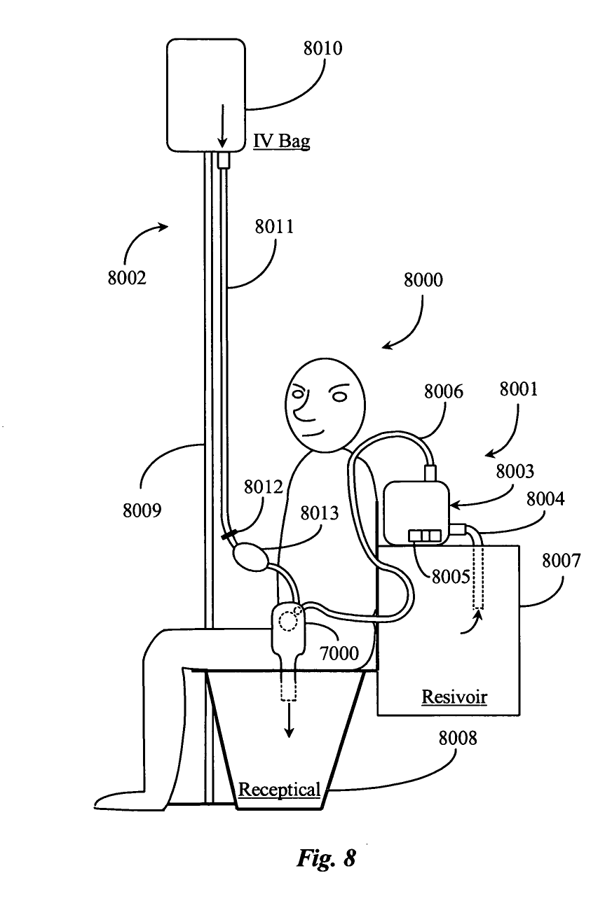

FIG. 8 is an elevation view of a user 8000 practicing colostomy evacuation and rinsing according to other embodiments of the present invention. User 8000 is illustrated in a seated position and wearing a colostomy bag analogous to colostomy bag 7000 described above. In actual practice, colostomy bag 7000 with the elongated bottleneck hangs down and between the user's legs. However, the left leg of the user is not illustrated herein to support a better view of bag 7000 and to aid description.

In one embodiment of the present invention, a pump system 8001 is provided and is adapted to deliver the aqueous solution into colostomy bag 7000. System 8001 includes a small electric pump 8003, which may be similar to one used in an aquarium filter system. In this case, filters and other apparatus are not required. Pump 8003 has an on/off switch 8005 for powering the device on and off. Pump 8003 may be mounted to the top of a reservoir 8007 typically provided to hold water for toilet flushing. In one embodiment, pump 8003 may simply rest on top of reservoir 8007, or it may be mounted to some other fixture nearby. Pump 8003 has an inlet tube 8004 adapted for the purpose of taking in water when the pump is activated. Inlet tube 8004 may be any standard plastic tubing that fits to an inlet valve opening on the pump. Tube 8004 is strategically placed inside reservoir 8007 below the water line so that water may pass into it. In an embodiment where pump 8003 is mounted according to an alternate configuration and perhaps location relative to reservoir 8007, tube 8004 may be an elongated tube to meet any length requirement.

Pump 8003 has an outlet tube 8006 fastened to an outlet valve of the pump. Tube 8006, like tube 8004 may be a standard plastic tubing size typically provided with an aquarium pump. One end of tube 8006 is modified to include a threaded female nozzle analogous in description to nozzle 3002 of syringe 3001 described with reference to FIG. 3. Tube 8006 is adapted in this case as a delivery tube for delivering aqueous solution into bag 7000. In this example, user 8000 wearing bag 7000 may take a seat on receptacle 8008 and may connect the free end of tube 8006 to the valve body on bag 7000. Connection may be by threading as previously described. When the user is ready. He or she simply switches on the pump for a limited amount of time to pump the aqueous solution from reservoir 8007 according to the direction of the arrow through pump 8003 and delivery tube 8006 and into bag 7000. The first introduction may be enough to enable breakdown of any solids into a liquid suitable for evacuation. It is noted herein that colostomy bag 7000 would be clamped at its opening likely for the duration of the first application of solution for breaking down solids. The matter is subsequently evacuated from bag 7000 directly into receptacle 8008 when the bag opening is unclamped, the liquefied contents flowing into the receptacle according to the direction of the arrow.

In one embodiment, pump 8003 has a safety switch setting that enable only so much solution to be delivered into bag 7000 at any one time. In this way a user has some protection against the prospect of delivering more water than the bag can contain based on its size and the amount of matter contained in the bag. In any case, once the solution is delivered into the bag, the user may externally work the bag now containing matter and solution to break down solids thereby “liquefying the contents” of bag 7000. At this point, the user may unclamp the opening of Bag 7000 to let the wastewater evacuate into the toilet receptacle. Once this has been accomplished, the user may activate switch 8005 successively to provide a rinsing stream of solution.

In one embodiment of the present invention, user 8000 may use the pump system 8001 for home use and syringe 3001 described with reference to FIG. 3 instead when he or she is traveling and not resident. Pump system 8001 may also be available in hospitals and the like for users who may require them. The fact that valve assembly 2007 described with respect to FIG. 2 only enables a one-way flow of gas or solution there is a low risk of contamination of any elements of the pump system such as the treaded end of tube 8006 for example. Pump system 8001 may also be used at a patient's bedside with any type of waste receptacle like a bedpan or a disposable vessel and a reservoir of clean water in place of the flush-tank reservoir.

In another embodiment of the present invention, a gravity evacuation system 8002 is provided as an alternative to pumping system 8001. In this embodiment, an IV bag 8010 is suspended a sufficient distance above user 8000 on an IV stand 8009. IV bag 8010 is sufficiently large to contain a significant amount of water or aqueous solution for the purpose. In this example, a delivery tube 8011 is provided that ports from the IV bag. Gravity forces water into the delivery tube. Tube 8011 may be a standard IV delivery tube made of medical grade plastic. In this embodiment, IV tube 8011 is clamped off using any type of hose clamp illustrated herein as a clamp 8012. Solution may travel via gravity down to the location of clamp 8012.

In one embodiment, tube 8011 includes a hollow bodied squeeze bulb 8013 adapted to contain solution and to eject solution by forcibly squeezing it out of the bulb. Bulb 8013 may be a contiguously formed member of tube 8011. The end of tube 8011 opposite IV bag 8010 may be threaded onto the valve assembly of bag 7000 identically as described with respect to tube 8006 of system 8001.

To use system 8002, user 8000 threads tube 8011 to bag 7000 and then unclamps clamp 8012. Squeeze bulb 8013 then fills with solution along with the rest of tube 8011 down to the valve on bag 7000. User 8000 may then clamp shut tube 8011 using clamp 8012. This action causes the solution to remain inside of the remainder of tube 8011 (section below clamp 8012). User 8000 may then forcibly squeeze bulb 8013 to force solution into bag 7000 through valve 2007 (described further above). At this point, bag 7000 is likely clamped at its bottom opening so that any solids may be broken down with the aid of the introduced solution.

If another delivery of solution is required, then user 8000 releases clamp 8012 to enable more solution to enter bulb 8013 and the remainder of tube 8011. User 8000 may then re-apply clamp 8012 and squeeze bulb 8013 to force another volume of solution into bag 7000. Again as previously noted, once the contents of bag 7000 are sufficiently liquefied, user 8000 may unclamp the opening at the bottom of bag 7000 and release the contents. Subsequent cycles may be repeated for top-down bag rinsing while user 8000 is still seated.

The cleaning or rinsing solution may be different in composition than the evacuation solution. For example, the evacuation solution may be water, while the cleaning solution may be water with a small amount of antibacterial agent introduced. In one case, there may be more than one IV bag and delivery tube if two different types of solution are available, for example, one to evacuate and another for rinsing. There are many possibilities.

In still another embodiment, IV gravity system 8002 may be provided without squeeze bulb 8013. In this case, the height of stand 8010 should be sufficient to force the solution into bag 7000 without requiring further valve modification. However, valve openings may be slightly enlarged to accommodate a more forceful flow of solution, and/or tube 3004 referenced with respect to FIG. 3 above may be elongated to further collapse plug 3009 to improve flow. The exact force of gravity acting on system 8002 is determined in part by the volume of solution in bag 8010 and by the height that it is suspended. Squeeze bulb 8013 simply provides a way to amplify the force of introduction of the solution.

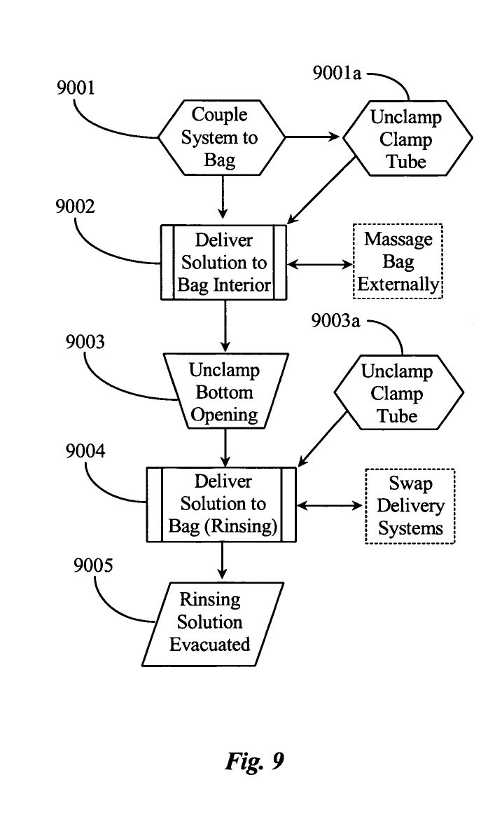

FIG. 9 is a process flow chart 9000 illustrating acts for evacuating and rinsing a colostomy bag through a colostomy bag valve according to an embodiment of the present invention. In act 9001, a solution delivery system is coupled to the colonostomy bag valve. In this act, the delivery system may be a manual injection system like a syringe or hollow bodied bulb. The delivery system may also be a pump system like a modified aquarium pump and connecting hose, or a suspended gravity system like an IV bag suspended on an IV stand.

In act 9002, an amount of aqueous solution is delivered through the valve on the colostomy bag. In this act, the method of delivery may be by a syringe in which case a portion or all of the contents of the syringe may be delivered in this act. In the case of a squeeze bulb delivery process flow 9000 may include a sub act 9001 a of unclamping and then clamping a delivery tube connected to the squeeze bulb at a point above the squeeze bulb to enable the solution to occupy the tube and bulb below the clamp position on the delivery tube before delivery. In both embodiments, the solution is forced through the valve and into the interior of the colostomy bag by force initiated and controlled by a user.

In one embodiment, act 9002 includes switching on and off a pump-activated delivery system, in which case the solution is delivered via mechanical force. In act 9003, the user of act 9002 may optionally massage the colostomy bag for the purpose aiding in breaking up any solid matter within the bag.

At act 9003, the user may unclamp the colostomy bag at the bottom opening to enable gravity evacuation of the bag contents into a receptacle. It is noted herein that act 9002, including any associated sub acts may be repeated one or more times before act 9003. In act 9004 more solution, or in one aspect, a separate solution may be delivered for the purpose of rinsing the interior of the colostomy bag and may also include any associated sub acts such as an act 9003a for clamping and unclamping the associated delivery tube. In one embodiment, there are 2 separate solutions. One solution may be maintained at ready for bag evacuation and one may be maintained at ready for bag rinsing and disinfecting. In the case of a syringe delivery system a user may swap out syringes (removing one and installing the next one) the next one adapted for rinsing. Likewise, a gravity-based delivery system like an IV system may also include 2 separate IV bags and delivery tubes, in which case swapping delivery systems may be pursued.

At act 9005, the rinsing solution evacuates via gravity through the bottom opening. It is noted herein that act 9004 for rinsing may be repeated more than one time in the case of measured delivery of solution such as via a syringe or squeeze bulb. One with skill in the art will recognize that the exact number and order of acts illustrated in flow chart 9000 may vary slightly according to different embodiments using different delivery systems and apparatus of the invention without departing from the spirit and scope of the present invention, some of those variations already described above. Other variations are also possible without departing from the scope of the present invention such as use of a squirt bottle instead of a syringe, the squirt bottle adapted with a short delivery tube modified to couple with the valve assembly of the colostomy bag.

Although it is preferred in most embodiments that a one-way valve is used that prevents gaseous order or material from escaping the colostomy bag, it is not specifically required in order to practice the present invention as two-way valves with a leak tight capping system may also be used. In the latter case the system may be restricted for use to one user to avoid any possibility of cross contamination that may be caused by material escaping from the two-way valve during the coupling process. In the light of the personal and temporary use of existing colostomy bags (typically ordered for and used by only one person), there should be no obstacle toward an embodiment with a two-way valve.

The methods and apparatus of the present invention may be practiced according to varied embodiments. For example, a user may practice the invention at home, at the hospital, or while in transit between locations. Existing colostomy bags may be easily modified by installing a suitable valve assembly in order to practice the present invention or new colostomy bags may be manufactured for the purpose with a valve assembly to practice the present invention. The spirit and scope of the present invention should therefore be given the broadest possible interpretation in light of the many possible uses and embodiments. The spirit and scope of the present invention is therefore limited only by the claims, which follow.

What is claimed is:

1. A system for top-down evacuating and rinsing of a user-mounted colostomy bag comprising:
a valve assembly affixed through one wall of the colostomy bag, the valve assembly having a first coupling interface and an interiorly protruding stem;
at least one solution reservoir for containing an aqueous solution; and
a second coupling interface attached directly to or indirectly to at least one of the at least one solution reservoir, the coupling interface for connecting the reservoir to the valve for communication of the aqueous solution from the reservoir to the bag.

2. The system of claim 1, wherein the valve assembly is a unidirectional flow valve that is air tight when not coupled.

3. The system of claim 1, wherein the exterior coupling interface is a valve body having an outside diameter thread formed thereon and the second coupling interface is a tube having an inner diameter thread formed thereon.

4. The system of claim 1, wherein the at least one solution reservoir is a syringe.

5. The system of claim 1, wherein the at least one solution reservoir is a suspended intravenous bag.

6. The system of claim 1, wherein the at least one solution reservoir is a toilet flush tank.

7. The system of claim 1, wherein the at least one solution reservoir is a squirt bottle.

8. The system of claim 1, wherein indirect attachment of the second coupling interface to the at least one solution reservoir is made by inclusion of a length of tubing between the connector and the reservoir.

9. The system of claim 8, wherein the reservoir is an intravenous bag.

10. The system of claim 9, further including:
a squeeze bulb for urging the aqueous solution forward and,
a tubing clamp for stopping and starting gravitational occupation of aqueous solution through the length of tubing.

11. A colostomy bag comprising:
at least two bag walls seamed together; and
a valve assembly permanently affixed through one of the bag walls, the assembly including an externally protruding coupling interface and an interiorly protruding valve stem.

12. The colostomy bag of claim 11, wherein the valve assembly couples to a system for delivering an aqueous solution into the bag while worn by a user.

13. The colostomy bag of claim 11, wherein the valve assembly is a unidirectional flow valve that is airtight when not coupled.

14. The colostomy bag of claim 12, wherein the externally protruding coupling interface is a valve body having an outside diameter thread formed thereon connectable to a second coupling interface having an inner diameter thread formed thereon.

15. A method for top-down evacuating and rinsing of a colostomy bag mounted to a user, the colostomy bag including a valve assembly permanently affixed through one of the bag walls, the assembly including an externally protruding coupling interface and an interiorly protruding valve stem including acts for:
(a) coupling a delivery system adapted to deliver an aqueous solution to the coupling interface;
(b) delivering an amount of the aqueous solution into the bag through the valve assembly;
(c) massaging the bag externally to break down solid matter into the solution;
(d) unclamping an evacuation opening at the bottom of the bag; and
(e) rinsing the bag by delivering an additional amount of aqueous solution into the bag through the valve.

16. The method of claim 15, wherein in act (a), the delivery system is a syringe coupled to the valve assembly by threading.

17. The method of claim 15, wherein in act (b), the valve assembly is a unidirectional flow valve that is airtight when not coupled.

18. The method of claim 15, wherein in act (a), the delivery system is a suspended intravenous bag indirectly coupled to the valve assembly via a length of tubing.

19. The method of claim 18 wherein the tubing includes a hollow-bodied squeeze bulb.

20. The method of claim 15, wherein in act (a), the delivery system is pump activated.
